(19) United States
(12) Patent Application Publication
(10) Pub. No.: US 2025/0072869 A1
(43) Pub. Date: Mar. 6, 2025

(54) ULTRASONIC DIAGNOSTIC APPARATUS, METHOD, AND STORAGE MEDIUM

(71) Applicant: CANON MEDICAL SYSTEMS CORPORATION, Tochigi (JP)

(72) Inventor: Minori OHASHI, Otawara (JP)

(73) Assignee: CANON MEDICAL SYSTEMS CORPORATION, Tochigi (JP)

(21) Appl. No.: 18/823,211

(22) Filed: Sep. 3, 2024

(30) Foreign Application Priority Data
Sep. 4, 2023 (JP) 2023-142883

(51) Int. Cl. A61B 8/00; A61B 8/06; A61B 8/08

(52) U.S. Cl. CPC A61B 8/463; A61B 8/06; A61B 8/488; A61B 8/5207

(57) ABSTRACT

An ultrasonic diagnostic apparatus according to an embodiment includes processing circuitry. The processing circuitry tracks a position of a part where a sample gate is set to designate a position of a part where Doppler information is collected. The processing circuitry performs control to display information about accuracy of tracking of the position of the part described above.

### CROSS-REFERENCE TO RELATED APPLICATIONS

This application is based upon and claims the benefit of priority from Japanese Patent Application No. 2023-142883, filed on Sep. 4, 2023; the entire contents of which are incorporated herein by reference.

### FIELD Embodiments described herein relate generally to an ultrasonic diagnostic apparatus, a method, and a storage medium.

### BACKGROUND

In the related art, ultrasonic diagnostic apparatus generate and display a Doppler spectrum (Doppler waveform) indicating blood flow rate information by using Doppler information (Doppler signal) extracted from reflected waves of ultrasonic waves. The Doppler waveform is a waveform obtained by plotting a blood flow rate within a range set by an operator as an observation part on a time-series basis. Such a range is set by the operator who refers to a two-dimensional ultrasonic image (a two-dimensional B-mode image, or a two-dimensional color Doppler image).

For example, in a PW mode for collecting Doppler waveforms using a Pulsed Wave (PW) Doppler method, the operator disposes a sample gate at a specific part in a blood vessel in accordance with running of the blood vessel depicted in the two-dimensional ultrasonic image. In the PW mode, a Doppler waveform indicating blood flow rate information in the sample gate is displayed.

The sample gate described above may be deviated from the part set by the operator due to influence of body motion and the like. Thus, as a method for coping with such deviation, there is known a method of tracking the sample gate using template matching. In this method, for example, by performing template matching on the two-dimensional ultrasonic image that is collected in real time after the sample gate is set, the part where the sample gate is set is tracked, and the position of the sample gate is moved.

### BRIEF DESCRIPTION OF THE DRAWINGS

FIG. 1 is a block diagram illustrating an example of a configuration of an ultrasonic diagnostic apparatus according to a first embodiment;

FIG. 2 is a flowchart illustrating a processing procedure of the ultrasonic diagnostic apparatus according to the first embodiment;

FIG. 3 is a diagram illustrating an example of setting of a sample gate according to the first embodiment; and

FIG. 4 is a diagram illustrating a display example according to the first embodiment.

### DETAILED DESCRIPTION

An ultrasonic diagnostic apparatus according to an embodiment includes processing circuitry. The processing circuitry is configured to track a position of a part where a sample gate is set to designate a position of a part where Doppler information is collected. The processing circuitry is configured to perform control to display information about accuracy of tracking of the position of the part described above.

The following describes an embodiment of an ultrasonic diagnostic apparatus, a method, and a storage medium according to the present application in detail with reference to the attached drawings. The ultrasonic diagnostic apparatus, the method, and the storage medium according to the present application are not limited to the embodiment described below.

### First Embodiment

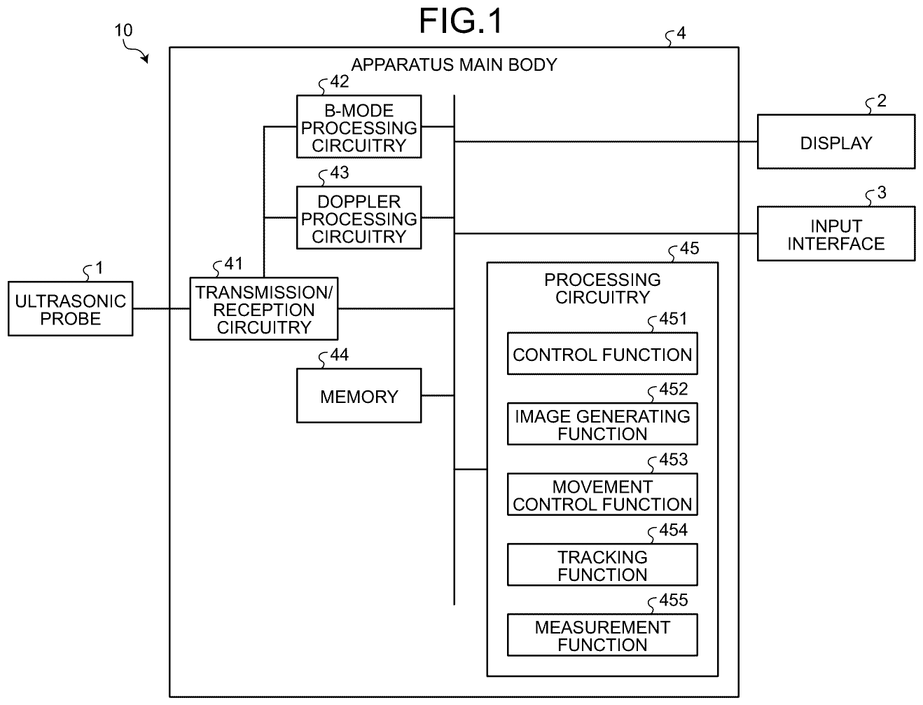

FIG. 1 is a block diagram illustrating an example of a configuration of an ultrasonic diagnostic apparatus 10 according to a first embodiment. As illustrated in FIG. 1, the ultrasonic diagnostic apparatus 10 according to the present embodiment includes an ultrasonic probe 1, a display 2, an input interface 3, and an apparatus main body 4. The ultrasonic probe 1, the display 2, and the input interface 3 are connected to the apparatus main body 4 in a communicable manner.

The ultrasonic probe 1 is connected to transmission/reception circuitry 41 included in the apparatus main body 4. The ultrasonic probe 1 includes, for example, a plurality of piezoelectric transducer elements in a probe main body, and these piezoelectric transducer elements generate ultrasonic waves based on a drive signal supplied from the transmission/reception circuitry 41. The ultrasonic probe 1 receives reflected waves from a subject, and converts them into electric signals. The ultrasonic probe 1 also includes, in the probe main body, a matching layer disposed in the piezoelectric transducer element, a backing material that prevents ultrasonic waves from propagating rearward from the piezoelectric transducer element, and the like. The ultrasonic probe 1 is connected to the apparatus main body 4 in a detachable manner. For example, the ultrasonic probe 1 is an ultrasonic probe of a sector type, a linear type, a convex type, or the like.

When ultrasonic waves are transmitted from the ultrasonic probe 1 to the subject, the transmitted ultrasonic waves are successively reflected by a discontinuous surface of acoustic impedance in body tissues of the subject, and received by the piezoelectric transducer elements included in the ultrasonic probe 1 as reflected wave signals. Amplitude of the reflected wave signal to be received depends on a difference in acoustic impedance on the discontinuous surface by which the ultrasonic waves are reflected. In a case in which transmitted ultrasonic pulses are reflected by a surface of a cardiac wall and the like or a moving blood flow, the reflected wave signals are subjected to frequency shift depending on a velocity component with respect to an ultrasonic wave transmitting direction of a mobile object due to the Doppler effect.

The ultrasonic probe 1 may be a one-dimensional ultrasonic probe in which the piezoelectric transducer elements are disposed in a line, an ultrasonic probe that mechanically oscillates the piezoelectric transducer elements in the one-dimensional ultrasonic probe, or a two-dimensional ultrasonic probe in which the piezoelectric transducer elements are two-dimensionally disposed in a grid-like fashion.

Herein, the ultrasonic diagnostic apparatus 10 according to the present embodiment collects Doppler waveforms using a Pulsed Wave (PW) Doppler method. In the present embodiment, the ultrasonic probe 1 connected to the apparatus main body 4 is an ultrasonic probe that can perform transmission/reception of ultrasonic waves for photographing B-mode image data and color Doppler image data, and transmission/reception of ultrasonic waves for collecting Doppler waveforms in the PW mode using the PW Doppler method.

The display 2 displays a Graphical User Interface (GUI) for the operator of the ultrasonic diagnostic apparatus 10 to input various setting requests using the input interface 3, an ultrasonic image generated in the apparatus main body 4, and the like. The display 2 also displays various kinds of messages and display information to notify the operator of a processing state and a processing result of the apparatus main body 4. The display 2 includes a speaker, and can output voice.

The input interface 3 is operated to perform setting and the like of a predetermined position (for example, a position of the sample gate and the like), and implemented, for example, by a trackball, a switch button, a mouse, a keyboard, a touch pad for performing an input operation by touching an operation surface, a touch monitor obtained by integrating a display screen with a touch pad, a noncontact input circuit using an optical sensor, a voice input circuit, and the like. The input interface 3 is connected to processing circuitry 45 (described later), and converts an input operation received from the operator into an electric signal to be output to the processing circuitry 45. In the present specification, the input interface 3 does not necessarily include a physical operation component such as a mouse and a keyboard. For example, examples of the input interface include a processing circuit for electric signals that receives an electric signal corresponding to an input operation from an external input appliance that is disposed separately from the apparatus, and outputs this electric signal to the processing circuitry 45.

The apparatus main body 4 includes the transmission/reception circuitry 41, B-mode processing circuitry 42, Doppler processing circuitry 43, a memory 44, and the processing circuitry 45. In the ultrasonic diagnostic apparatus 10 illustrated in FIG. 1, each of processing functions is stored in the memory 44 in a form of a computer program executable by a computer. Each of the transmission/reception circuitry 41, the B-mode processing circuitry 42, the Doppler processing circuitry 43, and the processing circuitry 45 is a processor that reads out, from the memory 44, and executes each computer program to implement a function corresponding to the computer program. In other words, each circuitry that has read out each computer program has a function corresponding to the read-out computer program.

The transmission/reception circuitry 41 includes a pulse generator, a transmission delay circuit, a pulser, and the like, and supplies a drive signal to the ultrasonic probe 1. The pulse generator repeatedly generates rate pulses for forming transmission ultrasonic waves at a predetermined rate frequency. The transmission delay circuit gives, to each rate pulse generated by the pulse generator, a delay time for each piezoelectric transducer element required for converging ultrasonic waves generated from the ultrasonic probe 1 in a beam shape and determining transmission directivity. The pulser applies a drive signal (drive pulse) to the ultrasonic probe 1 at a timing based on the rate pulse. That is, by changing the delay time given to each rate pulse, the transmission delay circuit optionally adjusts a transmitting direction of ultrasonic waves transmitted from a surface of the piezoelectric transducer element.

The transmission/reception circuitry 41 has a function capable of instantaneously changing a transmission frequency, a transmission driving voltage, and the like for performing a predetermined scanning sequence based on an instruction from the processing circuitry 45 (described later). Specifically, change of the transmission driving voltage is achieved by a transmission circuit of a linear amplifier type that can instantaneously change a value of the transmission driving voltage, or a mechanism that electrically switches between a plurality of power supply units.

The transmission/reception circuitry 41 includes a preamplifier, an Analog/Digital (A/D) converter, a reception delay circuit, an adder, and the like, and generates reflected wave data by performing various kinds of processing on the reflected wave signals received by the ultrasonic probe 1. The preamplifier amplifies the reflected wave signal for each channel. The A/D converter A/D converts the amplified reflected wave signal. The reception delay circuit gives a delay time required for determining reception directivity. The adder performs addition processing on the reflected wave signals processed by the reception delay circuit to generate reflected wave data. A reflection component from a direction corresponding to the reception directivity of the reflected wave signal is emphasized through the addition processing by the adder, and a comprehensive beam of transmission/reception of ultrasonic waves is formed based on the reception directivity and the transmission directivity.

The B-mode processing circuitry 42 receives the reflected wave data from the transmission/reception circuitry 41, and performs logarithmic amplification, envelope detection processing, and the like to generate data (B-mode data) representing signal strength as a degree of luminance.

The Doppler processing circuitry 43 performs frequency analysis on velocity information in the reflected wave data received from the transmission/reception circuitry 41, extracts a blood flow, tissues, and a contrast medium echo component due to the Doppler effect, and generates data (Doppler data) by extracting mobile object information such as velocity, dispersion, power, and the like for multiple points. For example, the mobile object is a fluid such as blood flowing in a blood vessel, or lymph flowing in a lymphatic vessel.

The B-mode processing circuitry 42 and the Doppler processing circuitry 43 can process both of two-dimensional reflected wave data and three-dimensional reflected wave data. That is, the B-mode processing circuitry 42 generates two-dimensional B-mode data from the two-dimensional reflected wave data, and generates three-dimensional B-mode data from the three-dimensional reflected wave data. The Doppler processing circuitry 43 generates two-dimensional Doppler data from the two-dimensional reflected wave data, and generates three-dimensional Doppler data from the three-dimensional reflected wave data.

The B-mode processing circuitry 42 can also generate the three-dimensional reflected wave data by combining a plurality of pieces of the two-dimensional reflected wave data, and generate the three-dimensional B-mode data from the generated three-dimensional reflected wave data. The Doppler processing circuitry 43 can also generate the three-dimensional reflected wave data by combining a plurality of pieces of the two-dimensional reflected wave data, and generate the three-dimensional Doppler data from the generated three-dimensional reflected wave data.

The memory 44 stores an ultrasonic image for display generated by the processing circuitry 45. The memory 44 can also store the B-mode data generated by the B-mode processing circuitry 42 and the Doppler data generated by the Doppler processing circuitry 43. The memory 44 also stores a control program for performing transmission/reception of ultrasonic waves, image processing, and display processing, diagnostic information (for example, a patient ID, findings of a doctor, and the like), and various kinds of data such as a diagnostic protocol and various body marks. The memory 44 also stores various kinds of information related to template matching (described later).

The processing circuitry 45 controls the entire processing of the ultrasonic diagnostic apparatus 10. Specifically, the processing circuitry 45 performs various kinds of processing by reading out, from the memory 44, and executes computer programs corresponding to a control function 451, an image generating function 452, a movement control function 453, a tracking function 454, and a measurement function 455 illustrated in FIG. 1. For example, the processing circuitry 45 is a processor that implements a function corresponding to each computer program by reading out, from the memory 44, and executing the computer program. In other words, the processing circuitry 45 that has read out each computer program has each function illustrated in the processing circuitry 45 in FIG. 1.

Herein, the control function 451 is an example of an acceptance unit and a display control unit. The movement control function 453 is an example of a movement control unit. The tracking function 454 is an example of a tracking unit. The measurement function 455 is an example of a measurement function. In the present embodiment, it is assumed that each of the processing functions described below is implemented by the single processing circuitry 45, but the processing circuitry may be configured by combining a plurality of independent processors, and each of the processors may execute a computer program to implement a function.

The control function 451 controls processing of the transmission/reception circuitry 41, the B-mode processing circuitry 42, and the Doppler processing circuitry 43 based on various setting requests input by the operator via the input interface 3, and various control programs and various kinds of data read from the memory 44. The control function 451 performs control to cause the display 2 to display an ultrasonic image and various kinds of information. For example, the control function 451 accepts a setting operation for setting the sample gate to designate a position of a part where the Doppler information is collected. The control function 451 also performs control to display information about accuracy of tracking of the position of the part.

The image generating function 452 generates an ultrasonic image from the data generated by the B-mode processing circuitry 42 and the Doppler processing circuitry 43. That is, the image generating function 452 generates an ultrasonic image in which intensity of the reflected wave is represented as luminance from the B-mode data generated by the B-mode processing circuitry 42. The image generating function 452 also generates an ultrasonic image representing the mobile object information (blood flow information or movement information of tissues) from the Doppler data generated by the Doppler processing circuitry 43. The ultrasonic image based on the Doppler data is velocity image data, dispersion image data, power image data, or image data combining them.

Herein, the image generating function 452 generally converts (scan-converts) a string of scanning line signals of ultrasonic scanning into a string of scanning line signals in a video format represented by a television and the like, and generates an ultrasonic image for display. Specifically, the image generating function 452 generates the ultrasonic image for display by performing coordinate transformation in accordance with a scanning mode for the ultrasonic waves by the ultrasonic probe 1. Additionally, as various kinds of image processing other than scan conversion, the image generating function 452 performs, for example, by using a plurality of image frames after scan conversion, image processing (smoothing processing) for regenerating an average value image of luminance, image processing (edge emphasis processing) using a differential filter in the image, and the like. The image generating function 452 combines the ultrasonic image with character information of various parameters, a scale, a body mark, and the like.

Furthermore, the image generating function 452 generates three-dimensional B-mode image data by performing coordinate transformation on the three-dimensional B-mode data generated by the B-mode processing circuitry 42. An image generating function 452 also generates three-dimensional Doppler image data by performing coordinate transformation on the three-dimensional Doppler data generated by the Doppler processing circuitry 43. Furthermore, to generate various two-dimensional images for displaying these pieces of three-dimensional image data (volume data) on the display 2, the image generating function 452 can perform rendering processing on the volume data.

Furthermore, the image generating function 452 can generate M-mode image data from time series data of the B-mode data on one scanning line generated by the B-mode processing circuitry 42. The image generation unit 14 can generate a Doppler waveform by plotting velocity information of a blood flow on a time-series basis from the Doppler data generated by the Doppler processing circuitry 43.

The movement control function 453 sets the sample gate on the two-dimensional ultrasonic image based on the setting operation for the sample gate accepted by the control function 451. The movement control function 453 moves the position of the sample gate based on a processing result obtained by the processing circuitry 45. Details about the processing performed by the movement control function 453 will be described later.

The tracking function 454 tracks the position of the part where the sample gate is set. Specifically, the tracking function 454 tracks the position of the part where the sample gate is set by template matching using the two-dimensional ultrasonic image at the time of collecting the Doppler information. Details about the processing performed by the tracking function 454 will be described later.

The measurement function 455 performs measurement processing for the Doppler waveform based on the Doppler information. Details about the processing performed by the measurement function 455 will be described later.

The configuration of the ultrasonic diagnostic apparatus 10 according to the first embodiment has been described above. With such a configuration, the ultrasonic diagnostic apparatus 10 improves diagnosis efficiency. Specifically, by displaying information about accuracy of tracking at the time of tracking the position of the part where the sample gate is set, the ultrasonic diagnostic apparatus 10 improves diagnosis efficiency in a diagnosis based on the Doppler waveform.

For example, there is known a method of correcting the position of the sample gate that is shifted due to influence of body motion and the like by tracking the part where the sample gate is set and moving the position of the sample gate. However, in this method, in a case in which tracking is incorrect or accuracy is insufficient, accuracy of diagnosis may be lowered, and as a result, the diagnosis efficiency may be lowered. Thus, the ultrasonic diagnostic apparatus 10 according to the present embodiment prevents accuracy of diagnosis from being lowered by displaying information about accuracy of tracking, and improves the diagnosis efficiency.

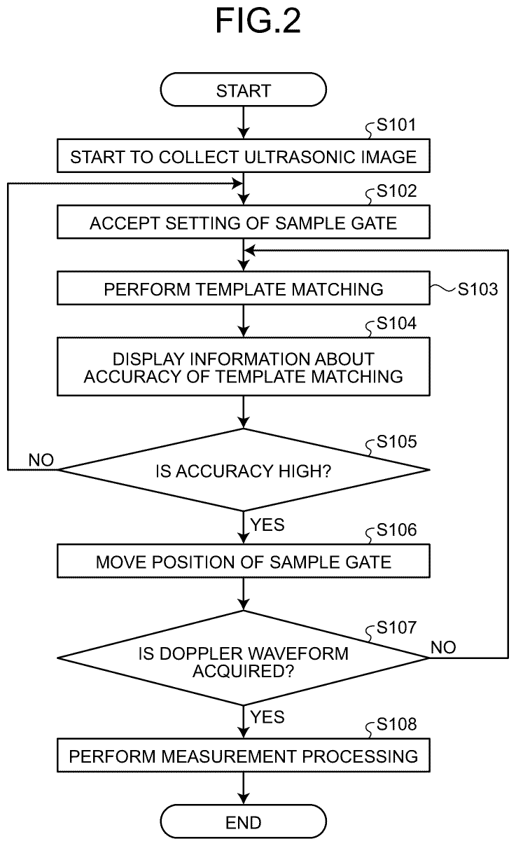

The following describes a processing procedure by the ultrasonic diagnostic apparatus 10 with reference to FIG. 2, and describes details about pieces of the processing thereafter. FIG. 2 is a flowchart illustrating the processing procedure of the ultrasonic diagnostic apparatus according to the first embodiment;

For example, as illustrated in FIG. 2, in the present embodiment, the control function 451 starts to collect the ultrasonic image (Step S101). Specifically, the control function 451 starts to collect the ultrasonic image in the PW mode in accordance with an operation via the input interface 3.

Subsequently, the control function 451 accepts setting of the sample gate for the collected ultrasonic image (Step S102). Specifically, the control function 451 accepts setting of the sample gate indicating a position for acquiring the Doppler waveform in accordance with an operation via the input interface 3. When the control function 451 accepts setting of the sample gate, the movement control function 453 sets the sample gate at the accepted position. Pieces of processing at Step S101 and S102 described above are implemented when the processing circuitry 45 calls, from the memory 44, and executes a computer program corresponding to the control function 451, for example.

Subsequently, the tracking function 454 performs template matching, which is targeted for the part where the sample gate is set, for the ultrasonic image being collected in real time (Step S103), and tracks the part where the sample gate is set. This processing is implemented when the processing circuitry 45 calls, from the memory 44, and executes a computer program corresponding to the tracking function 454, for example.

Subsequently, the control function 451 causes the display 2 to display information about accuracy of template matching (Step S104). This processing is implemented when the processing circuitry 45 calls, from the memory 44, and executes a computer program corresponding to the control function 451, for example.

Subsequently, the movement control function 453 determines whether the accuracy of tracking performed by the tracking function 454 is high (Step S105). Herein, if it is determined that the accuracy of tracking is high (Yes at Step S105), the movement control function 453 moves the position of the sample gate to the position specified by tracking (Step S106). On the other hand, if it is determined that the accuracy of tracking is not high at Step S105 (No at Step S105), the movement control function 453 does not move the sample gate, and returns the process to Step S102. The control function 451 then accepts setting of the sample gate by the operator. Pieces of processing at Step S105 and S106 described above are implemented when the processing circuitry 45 calls, from the memory 44, and executes a computer program corresponding to the movement control function 453, for example.

Subsequently, the measurement function 455 determines whether the Doppler waveform is acquired (Step S107). Specifically, the measurement function 455 determines whether the Doppler waveform required for performing measurement processing is acquired. Herein, if it is determined that the Doppler waveform is acquired (Yes at Step S107), the measurement function 455 performs measurement processing for the acquired Doppler waveform (Step S108), and ends the processing. On the other hand, if it is determined that the Doppler waveform is not acquired at Step S107 (No at Step S107), the process returns to Step S103, and the template matching is continuously performed by the tracking function 454.

In FIG. 2, described is a case of performing, after displaying the information about the accuracy of template matching at Step S104, processing of automatically moving the sample gate in accordance with the accuracy of template matching (Step S105 and Step S106), and automatic measurement processing for the acquired Doppler waveform (Step S107 and Step S108). However, the embodiment is not limited thereto. The processing of automatically moving the sample gate, and the automatic measurement processing for the Doppler waveform are not necessarily performed.

The following describes details about pieces of processing performed by the ultrasonic diagnostic apparatus 10.

### Collection of Ultrasonic Images

As described above at Step S101, the control function 451 starts to collect ultrasonic images in the PW mode. Herein, the control function 451 performs control to perform transmission/reception of ultrasonic waves for photographing the B-mode image data and the color Doppler image data along with transmission/reception of ultrasonic waves for collecting Doppler waveforms in the PW mode. That is, the control function 451 controls the transmission/reception circuitry 41 to collect two-dimensional ultrasonic images along with collection of the Doppler waveforms.

### Setting of Sample Gate

As described above at Step S101, the control function 451 accepts setting of the position of the sample gate in accordance with an operation via the input interface 3. The movement control function 453 then sets the sample gate at the position accepted by the control function 451. For example, the operator operates the input interface 3 to set the sample gate on the B-mode image data collected by the control function 451. The control function 451 notifies the movement control function 453 of position information of the sample gate accepted on the B-mode image data (coordinates on the B-mode image data). The movement control function 453 sets, as the position of the sample gate, the position information (coordinates on the B-mode image data) notified from the control function 451.

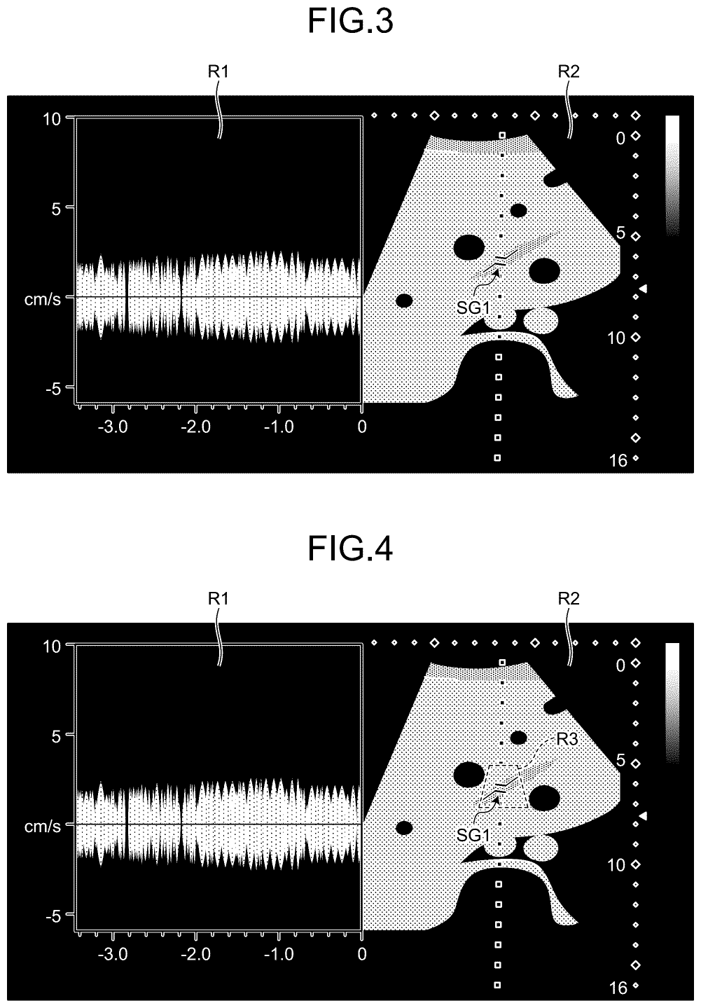

FIG. 3 is a diagram illustrating an example of setting of the sample gate according to the first embodiment. Herein, a diagram illustrated in a region R1 of FIG. 3 is the Doppler waveform displayed on the display 2. A diagram illustrated in a region R2 of FIG. 3 is the B-mode image data of a collection cross section displayed on the display 2. Specifically, the diagram illustrated in the region R2 of FIG. 3 is the B-mode image data of the collection cross section at which a blood vessel as an observation target is depicted in a long-axis cross section. The operator disposes a sample gate SG1 (parallel double lines illustrated on the right side of FIG. 3) in the long-axis cross section of the blood vessel in the B-mode image data.

The position information of the sample gate becomes position information that defines a collection range for sampling the Doppler information. The control function 451 acquires coordinates on the B-mode image data (coordinates in a scanning space for ultrasonic waves) corresponding to the position information of the sample gate set by the operator, and notifies the movement control function 453 of the coordinates. The movement control function 453 sets, as the position of the sample gate, the coordinates notified from the control function 451.

When the position of the sample gate is set, the control function 451 performs scanning in the PW mode under a scan condition for scanning the set position of the sample gate. The control function 451 then calculates blood flow rate information in a running direction of the blood vessel based on the Doppler information collected by scanning in the PW mode, generates the Doppler waveform indicating the calculated blood flow rate information, and causes the display 2 to display the Doppler waveform as illustrated in the region R1 of FIG. 3.

### Template Matching

As described above at Step S103, the tracking function 454 performs template matching, which is targeted for the part where the sample gate is set, for the ultrasonic image being collected in real time to track the part where the sample gate is set.

Specifically, the tracking function 454 first extracts, as a template image, a region in the vicinity of the set sample gate from the B-mode image data, and stores the extracted template image in the memory 44. Herein, the region extracted as the template image can be optionally set. For example, the tracking function 454 includes the set sample gate, and extracts, as the template image, a rectangular region formed of a predetermined number of pixels.

The tracking function 454 then performs template matching using the template image for newly collected B-mode image data as a target image to extract a region having the highest matching degree with the template image from the newly collected B-mode image data. Herein, the tracking function 454 can calculate the matching degree using a known calculation method (for example, squared difference, cross correlation, and the like).

As described above, after calculating the matching degree, the tracking function 454 associates the matching degree with the B-mode image data for which the matching degree is calculated, and stores it in the memory 44. That is, the tracking function 454 associates the matching degree of template matching with a target image of the template matching to be stored. Due to this, the matching degree of the template matching can be presented at the time of reading a collected image.

The template image may be updated by an image of the region having the highest matching degree with the template image. In such a case, for example, the tracking function 454 updates the template image to the image of the region having the highest matching degree in a case in which the matching degree is equal to or larger than a predetermined threshold.

### Display Processing for Information About Accuracy

As described above at Step S104, the control function 451 causes the display 2 to display the information about the accuracy of tracking performed by the tracking function 454. For example, the control function 451 causes the display 2 to display at least one of a numerical value indicating the accuracy of tracking, the sample gate the color of which is changed in accordance with the accuracy of tracking, and the Doppler waveform based on the Doppler information the color of which is changed in accordance with the accuracy of tracking.

By way of example, the control function 451 causes the display 2 to display the position of the part (region) having the highest matching degree in template matching in a discriminable manner, and display a numerical value indicating the matching degree. For example, the control function 451 causes a new sample gate having different color to be displayed at the position of the part (region) having the highest matching degree in template matching, and causes the numerical value indicating the matching degree to be displayed in the vicinity of the new sample gate. Due to this, the operator can determine whether to move the sample gate to the position of the sample gate that is newly displayed while referring to the numerical value indicating the matching degree.

For example, the control function 451 causes the sample gate and the Doppler waveform to be displayed while changing the color thereof in accordance with the matching degree of the part (region) having the highest matching degree in template matching. By way of example, in a case in which the matching degree is equal to or larger than the threshold when the matching degree calculated by the tracking function 454 is compared with the threshold, the control function 451 displays the sample gate and the Doppler waveform in a color indicating that the accuracy of template matching is high. On the other hand, in a case in which the matching degree is smaller than the threshold, the control function 451 displays the sample gate and the Doppler waveform in a color indicating that the accuracy of template matching is low. The threshold compared with the matching degree can be optionally set. Due to this, the operator can check the accuracy of tracking at the present time, and determine whether to move the sample gate.

### Movement Processing for Sample Gate

As described above at Steps S105 and S106, the movement control function 453 causes the position of the sample gate to automatically move in accordance with the accuracy of tracking. Specifically, in a case in which the matching degree is equal to or larger than the threshold when the matching degree at the part having the highest matching degree in template matching is compared with the threshold, the movement control function 453 moves the sample gate to the part having the highest matching degree in template matching. That is, the movement control function 453 causes the sample gate to automatically move to coordinates in a scanning space for ultrasonic waves corresponding to coordinates of the part having the highest matching degree in template matching in the newly collected B-mode image data.

The movement control function 453 can also cause the sample gate to move in accordance with an approval operation by the operator in a case in which the matching degree is equal to or larger than the threshold. In such a case, the control function 451 causes a new sample gate and a numerical value indicating the matching degree to be displayed at the position of the part (region) having the highest matching degree in template matching, and causes a GUI for approving movement of the sample gate to the position to be displayed. In a case in which the operator performs the approval operation via the GUI, the movement control function 453 causes the sample gate to move to the coordinates in the scanning space for ultrasonic waves corresponding to the coordinates of the part having the highest matching degree in template matching.

On the other hand, in a case in which the matching degree at the part having the highest matching degree in template matching is smaller than the threshold, the movement control function 453 does not cause the sample gate to automatically move, and causes the sample gate to move to the position corresponding to a movement operation for the sample gate by the operator. In such a case, for example, the control function 451 causes display information for prompting the operator to manually move the sample gate to be displayed because the accuracy of tracking is low while displaying the numerical value indicating the matching degree in template matching. In accordance with this display, when the operator resets the sample gate similarly to the first setting of the sample gate, the movement control function 453 causes the sample gate to move to the reset position.

The following describes an example of determination related to the movement processing for the sample gate. For example, as thresholds to be compared with the matching degree, “a”, “b”, and “c” are set (where a>b>c). The movement control function 453 compares the matching degree calculated by the tracking function 454 with “a”, “b”, and “c” to make determination related to the movement processing for the sample gate.

For example, in a case in which “matching degree<c” is satisfied, the movement control function 453 does not perform the movement processing for the sample gate, and makes determination to cause display information for prompting the operator to manually move the sample gate to be displayed because the accuracy of tracking is low.

For example, in a case in which “c<matching degree<b” is satisfied, the movement control function 453 makes determination to cause the sample gate to automatically move. At this point, the control function 451 notifies that the accuracy of tracking is low with a color of the sample gate or the Doppler waveform.

For example, in a case in which “a<matching degree” is satisfied, the movement control function 453 makes determination to cause the sample gate to automatically move. At this point, the control function 451 notifies that the accuracy of tracking is high with a color of the sample gate or the Doppler waveform.

As described above, the movement control function 453 causes the sample gate to move to a tracked position in a case in which the accuracy of tracking is high. However, it is desirable that the Doppler waveform is continuously acquired for one or more heartbeats, so that the movement control function 453 can perform control so that the sample gate is not easily moved.

For example, under the condition that a difference between the part having the highest matching degree in template matching and the part where the sample gate is set at the present time exceeds the threshold, the movement control function 453 causes the sample gate to move to the position of the part having the highest matching degree in template matching. That is, in a case in which the difference between the part having the highest matching degree in template matching and the part where the sample gate is set at the present time is equal to or smaller than the threshold, the movement control function 453 does not cause the sample gate to move. The difference between the part having the highest matching degree in template matching and the part where the sample gate is set at the present time is, for example, a distance between the parts.

Herein, the threshold to be compared with the difference described above is dynamically changed in accordance with at least one of a size of the sample gate, a width of an ultrasonic beam, and a frame rate. For example, as the size of the sample gate is smaller, the width of the ultrasonic beam is narrower, and the frame rate is higher, a smaller threshold is set. On the other hand, as the size of the sample gate is larger, the width of the ultrasonic beam is wider, and the frame rate is lower, a larger threshold is set.

Additionally, for example, the movement control function 453 causes the position of the sample gate to move under the condition that a predetermined time has elapsed after the sample gate is moved. That is, after moving the sample gate once, the movement control function 453 does not move the sample gate again until the predetermined time elapses.

Herein, the predetermined time described above is dynamically changed in accordance with at least one of the part, the frame rate, a heartbeat cycle, and a respiration cycle. For example, in a case in which the target is a part that is easily affected by respiration and body motion such as an abdominal region, a short time is set as the predetermined time. In a case in which the target is a part with less motion such as legs, a long time is set as the predetermined time. For example, as the frame rate is higher, the heartbeat cycle is shorter, and the respiration cycle is shorter, a shorter time is set as the predetermined time. As the frame rate is lower, the heartbeat cycle is longer, and the respiration cycle is longer, a longer time is set as the predetermined time.

### Display Processing Related to Movement Processing for Sample Gate

As described above, in a case in which a distance between the part having the highest matching degree in template matching and the part where the sample gate is set at the present time is equal to or smaller than the threshold, the movement control function 453 does not cause the sample gate to move. That is, the movement control function 453 does not cause the sample gate to automatically move in a predetermined range from the position where the sample gate is set at the present time.

In this case, the control function 451 can cause a range to which the sample gate is not moved to be displayed on the ultrasonic image. FIG. 4 is a diagram illustrating a display example according to the first embodiment. For example, as illustrated in FIG. 4, the control function 451 can cause a range R3 to which the sample gate is not moved to be overlapped with and displayed on the B-mode image data that has been collected at the present time. Herein, for example, the control function 451 can superimpose and display, on the B-mode image data, the range R3 that is not automatically followed in a fan shape for a convex probe or a sector probe, or in a rectangular shape or trapezoidal shape for a linear probe.

The control function 451 can also change a display form of the Doppler waveform based on the Doppler information in a time section related to movement of the sample gate. Specifically, the control function 451 changes the scan condition in a case in which the position of the sample gate is moved by the movement control function 453, but cannot acquire the Doppler waveform while the scan condition is being changed. Thus, in a time section in which the Doppler waveform cannot be acquired, for example, the control function 451 changes the color of the Doppler waveform displayed on the display 2. Due to this, the operator can immediately grasp that appropriate data is not acquired.

### Measurement Processing

As described above at Steps S107 and S108, the measurement function 455 determines whether the Doppler waveform is acquired, and automatically performs measurement processing on the acquired Doppler waveform. For example, the measurement function 455 determines whether the Doppler waveform has been continuously acquired for one or more heartbeats, and automatically performs various kinds of measurement processing on the Doppler waveform continuously acquired for one or more heartbeats.

Herein, the measurement function 455 can use accuracy of tracking by the tracking function 454 in determination at the time of performing the measurement processing described above. Specifically, the measurement function 455 performs measurement processing for a section in which the accuracy of tracking is relatively high, and a section in which the Doppler waveform is acquired. That is, the measurement function 455 performs various kinds of measurement processing on the Doppler waveform that has been continuously acquired for one or more heartbeats, the Doppler waveform in a section in which the accuracy of tracking is relatively high (for example, the matching degree in template matching is equal to or larger than the threshold). Due to this, more accurate results can be acquired in automatic measurement processing for the Doppler waveform.

As described above, according to the first embodiment, the control function 451 accepts a setting operation for setting the sample gate to designate the position of the part where the Doppler information is collected. The tracking function 454 tracks the position of the part where the sample gate is set. The control function 451 performs control to display information about the accuracy of tracking of the position of the part. Thus, the ultrasonic diagnostic apparatus 10 according to the first embodiment can present the accuracy of tracking to the operator, enable the operator to easily determine whether appropriate Doppler information is acquired, and improve diagnosis efficiency.

For example, a diagnosis can be performed while referring to the accuracy of tracking in a real-time diagnosis and a diagnosis by reading after collecting images, and the diagnosis efficiency can be improved.

According to the first embodiment, the tracking function 454 tracks the position of the part where the sample gate is set by template matching using the two-dimensional ultrasonic image at the time of collecting the Doppler information. Thus, the ultrasonic diagnostic apparatus 10 according to the first embodiment can present information about the accuracy of tracking by template matching.

According to the first embodiment, the control function 451 causes at least one of a numerical value indicating the accuracy of tracking, the sample gate the color of which is changed in accordance with the accuracy of tracking, and the Doppler waveform based on the Doppler information the color of which is changed in accordance with the accuracy of tracking to be displayed. Thus, the ultrasonic diagnostic apparatus 10 according to the first embodiment can cause the accuracy of tracking to be clearly displayed.

According to the first embodiment, the control function 451 displays the position of the part having the highest matching degree in template matching in a discriminable manner in the two-dimensional ultrasonic image. Thus, the ultrasonic diagnostic apparatus 10 according to the first embodiment can visually present the position having the highest matching degree.

According to the first embodiment, the movement control function 453 causes the sample gate to move to the position of the part having the highest matching degree in template matching in the two-dimensional ultrasonic image. The control function 451 changes the display form of the Doppler waveform based on the Doppler information in a time section related to movement of the sample gate. Thus, the ultrasonic diagnostic apparatus 10 according to the first embodiment can enable the Doppler waveform having low accuracy to be easily grasped.

According to the first embodiment, the measurement function 455 performs measurement processing on the Doppler waveform based on the Doppler information. The measurement function 455 performs measurement processing for a section in which the accuracy of tracking is relatively high, and a section in which the Doppler waveform is acquired. Thus, the ultrasonic diagnostic apparatus 10 according to the first embodiment can perform measurement processing with high accuracy.

According to the first embodiment, the movement control function 453 causes the sample gate to move to the position of the part having the highest matching degree in template matching in the two-dimensional ultrasonic image. The movement control function 453 causes the sample gate to move to the position of the part having the highest matching degree in template matching under the condition that a difference between the part having the highest matching degree in template matching and the part where the sample gate is set at the present time exceeds the threshold. Thus, the ultrasonic diagnostic apparatus 10 according to the first embodiment can prevent frequent update of the position of the sample gate, and can update the position of the sample gate in real time at a frequency of not hindering a diagnosis.

According to the first embodiment, the threshold is dynamically changed in accordance with at least one of the size of the sample gate, the width of the ultrasonic beam, and the frame rate. Thus, the ultrasonic diagnostic apparatus 10 according to the first embodiment can set an appropriate threshold for a difference between the part having the highest matching degree in template matching and the part where the sample gate is set at the present time.

According to the first embodiment, the control function 451 causes a range to which the sample gate is not moved to be displayed on the ultrasonic image. Thus, the ultrasonic diagnostic apparatus 10 according to the first embodiment can visually present a range in which the Doppler waveform can be accurately acquired.

According to the first embodiment, the movement control function 453 causes the sample gate to move to the position of the part having the highest matching degree in template matching in the two-dimensional ultrasonic image. The movement control function 453 causes the position of the sample gate to move under the condition that the predetermined time has elapsed after the sample gate is moved. Thus, the ultrasonic diagnostic apparatus 10 according to the first embodiment can update the sample gate at an appropriate timing.

According to the first embodiment, the predetermined time is dynamically changed in accordance with at least one of the part, the frame rate, the heartbeat cycle, and the respiration cycle. Thus, the ultrasonic diagnostic apparatus 10 according to the first embodiment can set an appropriate timing as a timing for updating the sample gate.

### Other Embodiments

In the embodiment described above, described is a case of causing the sample gate to automatically move in a case in which the accuracy of template matching (tracking) is high. However, the embodiment is not limited thereto. For example, the sample gate may be caused to automatically move irrespective of the accuracy of tracking. That is, at the time of ultrasonic scanning (real time), the accuracy of tracking may be only associated with corresponding B-mode image data to be stored while displaying information about the accuracy of tracking.

In the embodiment described above, described is a case in which the ultrasonic diagnostic apparatus 10 is an apparatus that sets a single sample gate. However, the embodiment is not limited thereto, and the ultrasonic diagnostic apparatus 10 may be an apparatus that sets a plurality of sample gates. In a case of such an ultrasonic diagnostic apparatus, an operation of the sample gate tends to be complicated. Thus, by applying the embodiment according to the present application to such an ultrasonic diagnostic apparatus, diagnosis efficiency can be further improved.

The embodiment described above can also be applied to a portable ultrasonic diagnostic apparatus. In many cases, the portable ultrasonic diagnostic apparatus does not include a device appropriate for an operation of the sample gate such as a trackball or a mouse. Thus, by applying the embodiment according to the present application to the portable ultrasonic diagnostic apparatus, operations related to the sample gate can be reduced, and the diagnosis efficiency can be further improved.

In the embodiment described above, described is a case of performing transmission/reception of ultrasonic waves for photographing the B-mode image data along with transmission/reception of ultrasonic waves for collecting the Doppler waveform in the PW mode, but the embodiment is not limited thereto. For example, only the B-mode image data for setting the sample gate may be collected first, and transmission/reception of ultrasonic waves for collecting the Doppler waveform in the PW mode and transmission/reception of ultrasonic waves for photographing the B-mode image data may be performed thereafter.

Alternatively, for example, only the B-mode image data for setting the sample gate may be collected, and thereafter, only the B-mode image data may be collected again to check the position of the sample gate, and information about the accuracy of tracking may be displayed.

In the embodiment described above, described is a case of using template matching for tracking the part where the sample gate is set. However, the embodiment is not limited thereto, and the part where the sample gate is set may be tracked by using other methods.

For example, the tracking function 454 tracks the part where the sample gate is set by extracting a region of the part where the sample gate is set every time the B-mode image data is collected. The tracking function 454 extracts the region of the part where the sample gate is set using a known region extraction technique (for example, a region expansion method, a snake method, a graph cut method, a mean shift method, and the like) or a machine learning technique.

For example, the tracking function 454 detects body motion of the subject by a position sensor attached to the subject or a camera image obtained by photographing the subject, and tracks the part where the sample gate is set based on the detected body motion.

A term of “processor” used in the above description means, for example, a Central Processing Unit (CPU), a Graphics Processing Unit (GPU), or a circuit such as an Application Specific Integrated Circuit (ASIC) and a programmable logic device (for example, a Simple Programmable Logic Device (SPLD), a Complex Programmable Logic Device (CPLD), and a Field Programmable Gate Array (FPGA)). The processor implements a function by reading out and executing a computer program stored in the memory. Instead of storing the computer program in the memory, the computer program may be directly incorporated into the circuit of the processor. In this case, the processor implements the function by reading out and executing the computer program incorporated in the circuit. Each processor according to the present embodiment is not necessarily configured as a single circuit for each processor. A plurality of independent circuits may be combined to be configured as one processor to implement the function.

The constituent elements of the apparatus illustrated in the drawings in the description of the above embodiment are merely conceptual, and it is not required that they are physically configured as illustrated necessarily. That is, specific forms of distribution and integration of the apparatus are not limited to those illustrated in the drawings. All or part thereof may be functionally or physically distributed/integrated in arbitrary units depending on various loads or usage states. Furthermore, all or optional part of processing functions executed by the respective apparatus may be implemented by a CPU and a computer program analyzed and executed by the CPU, or may be implemented as hardware using wired logic.

The method described in the above embodiment can be implemented by executing a computer program prepared in advance by a computer such as a personal computer or a workstation. This computer program can be distributed via a network such as the Internet. This computer program can also be executed by being recorded in a computer-readable non-transitory recording medium such as a hard disk, a flexible disk (FD), a CD-ROM, an MO, a DVD, or a Flash memory such as a USB memory and an SD card memory, and being read out from the non-transitory recording medium by the computer.

As described above, according to the embodiment, diagnosis efficiency can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
processing circuitry configured to
track a position of a part where a sample gate is set to designate a position of a part where Doppler information is collected, and
perform control to display information about accuracy of tracking of the position of the part.

2. The ultrasonic diagnostic apparatus according to claim 1, wherein the processing circuitry is configured to accept a setting operation for setting the sample gate.

3. The ultrasonic diagnostic apparatus according to claim 1, wherein the processing circuitry is configured to track the position of the part where the sample gate is set by template matching using a two-dimensional ultrasonic image at the time of collecting the Doppler information.

4. The ultrasonic diagnostic apparatus according to claim 1, wherein the processing circuitry is configured to cause at least one of a numerical value indicating the accuracy of tracking, a sample gate the color of which is changed in accordance with the accuracy of tracking, and a Doppler waveform based on the Doppler information the color of which is changed in accordance with the accuracy of tracking to be displayed.

5. The ultrasonic diagnostic apparatus according to claim 3, wherein the processing circuitry is configured to cause a display to display the position of the part having the highest matching degree in template matching in a discriminable manner in the two-dimensional ultrasonic image.

6. The ultrasonic diagnostic apparatus according to claim 3, wherein
the processing circuitry is configured to
cause the sample gate to move to the position of the part having the highest matching degree in template matching in the two-dimensional ultrasonic image, and
change a display form of the Doppler waveform based on the Doppler information in a time section related to movement of the sample gate.

7. The ultrasonic diagnostic apparatus according to claim 1, wherein
the processing circuitry is configured to
perform measurement processing on the Doppler waveform based on the Doppler information, and
perform the measurement processing for a section in which accuracy of the tracking is relatively high, and a section in which the Doppler waveform is acquired.

8. The ultrasonic diagnostic apparatus according to claim 3, wherein
the processing circuitry is configured to
cause the sample gate to move to the position of the part having the highest matching degree in template matching in the two-dimensional ultrasonic image, and
cause the sample gate to move to the position of the part having the highest matching degree in template matching under the condition that a difference between the part having the highest matching degree in template matching and the part where the sample gate is set at the present time exceeds a threshold.

9. The ultrasonic diagnostic apparatus according to claim 8, wherein the threshold is dynamically changed in accordance with at least one of a size of the sample gate, a width of an ultrasonic beam, and a frame rate.

10. The ultrasonic diagnostic apparatus according to claim 1, wherein the processing circuitry is configured to cause a range to which the sample gate is not moved to be displayed on an ultrasonic image.

11. The ultrasonic diagnostic apparatus according to claim 3, wherein
the processing circuitry is configured to
cause the sample gate to move to the position of the part having the highest matching degree in template matching in the two-dimensional ultrasonic image, and
cause the position of the sample gate to move under the condition that a predetermined time has elapsed after the sample gate is moved.

12. The ultrasonic diagnostic apparatus according to claim 11, wherein the predetermined time is dynamically changed in accordance with at least one of a part, a frame rate, a heartbeat cycle, and a respiration cycle.

13. A method comprising:
tracking a position of a part where a sample gate is set to designate a position of a part where Doppler information is collected; and
performing control to display information about accuracy of tracking of the position of the part.

14. A storage medium storing therein, in a non-transitory manner, a program that causes a computer to execute:
tracking a position of a part where a sample gate is set to designate a position of a part where Doppler information is collected; and
performing control to display information about accuracy of tracking of the position of the part.
